SEQUENCE LISTING (5 sequences)

SEQ ID NO: 1 - protein, 270 aa, Homo sapiens
MKPKMKYSTNKISTAKWKNTASKALCFKLGKSQQKAKEVCPMYFMKLRSGLMIKKEACYFRRETTKRPSLKTGRKHKRHLVLAACQQQSTVECFAFGISGVQKYTRALHDSSITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 2 - protein, 176 aa, Homo sapiens
AFGISGVQKYTRALHDSSITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 3 - protein, 172 aa, Homo sapiens
SGVQKYTRALHDSSITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 4 - protein, 162 aa, Homo sapiens
HDSSITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 5 - protein, 159 aa, Homo sapiens
SITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

(19) United States
(12) Patent Application Publication
(10) Pub. No.: US 2015/0259645 A1
(43) Pub. Date: Sep. 17, 2015

(54) METHOD FOR INDUCING IL-2-FREE PROLIFERATION OF GAMMA DELTA T CELLS

(71) Applicants: INSERM (INSTITUT NATIONAL DE LA SANTÉ ET DE LA RECHERCHE MÉDICALE), Paris (FR); CENTRE NATIONAL DE LA RECHERCHE SCIENTIFIQUE (CNRS), Paris (FR); UNIVERSITÉ PAUL SABATIER TOULOUSE III, Toulouse (FR)

(72) Inventors: Mary Poupot, Toulouse (FR); Jean-Jacques Fournie, Toulouse (FR); Caroline Duault, Toulouse (FR); Jean-Philippe Girard, Toulouse (FR); Corinne Cayrol-Girard, Toulouse (FR)

(73) Assignees: INSERM (INSTITUT NATIONAL DE LA SANTÉ ET DE LA RECHERCHE MÉDICALE, Paris (FR); CENTRE NATIONAL DE LA RECHERCHE SCIENTIFIQUE (CNRS), Paris (FR); UNIVERSITÉ PAUL SABATIER TOULOUSE III, Toulouse (FR)

(21) Appl. No.: 14/440,921

(22) PCT Filed: Nov. 8, 2013
(86) PCT No.: PCT/EP2013/073328

§ 371 Date: May 6, 2015

(30) Foreign Application Priority Data
Nov. 8, 2012 (EP) 12306385.1

(51) Int. Cl. C12N 5/0783; A61K 31/663; A61K 38/20

(52) U.S. Cl. CPC C12N 5/0636; A61K 38/20; A61K 31/663; C12N 2501/2333; C12N 2501/999

(57) ABSTRACT

The present invention concerns a method of inducing IL-2-free proliferation of γδ T cells using a combination of a γδ T cell activator and IL-33 for use in therapy of infection, cancer, autoimmunity as well as other diseases.

### FIELD OF THE INVENTION

The present invention relates to a method for inducing proliferation of γδ T lymphocytes using a combination of interleukin-33 (IL-33) and a γδ T cells activator for the treatment of an infectious disease or cancer therapy.

### BACKGROUND OF THE INVENTION

γδ T lymphocytes are known as non-conventional lymphocytes with respect to their characteristics at the interface of the innate and adaptive immunity. They recognize antigens with their TCR but without presentation or restriction by molecules of the complex major histocompatibility. The major subpopulation of γδ T lymphocytes in the human blood, the Vγ9Vδ2 T lymphocytes, recognizes in particular non peptidic antigens called phosphoantigens (PAgs). These PAgs are produced by some pathogen microorganisms and by human cancer cells (Poupot M, Fournie J J Immunol Lett 2004). By their ability to produce pro-inflammatory cytokines and the cytotoxicity induced upon their activation, these lymphocytes are very important actors of the antitumor immunity. They have indeed a high cytolitic potential in vitro against numerous cancer cell types such as established cancer cell lines or cells from cancer patients (renal or prostatic carcinoma or multiple myeloma) (Viey E et al. Immunol 2005; Liu Z et al. J Urol 2005; Kunzmann V et al. Blood 2000). Their cytolytic potential was also showed in vivo in immunodeficient mouse with human tumor xenografts (Kabelitz D et al. J Immunol 2004). Furthermore clinical trials based on the administration of phosphoantigens and IL2 showed an increase of the Vγ9Vδ2 T cells number in the blood of patients and a tumor reduction (Wilhelm M et al. Blood 2003; Bonneville M, Scotet E Curr Opin Immunol 2006).

Actually, if the Vγ9Vδ2 T lymphocytes represent only one percent of total lymphocytes in blood, the PAgs/IL-2 combination leads to the proliferation of these cells. It is moreover possible to considerably amplify this cellular population by growing PBMC (Peripheral Blood Mononuclear Cells) in vitro in the presence of PAgs and IL-2 to reach a purity of around 80%. Through this method several billions of cytotoxic Vγ9Vδ2 T lymphocytes can be obtained and subsequently re-injected in the patient for triggering an antitumor immunotherapy. The first phase I clinical trials performed showed a good safety of the grafts of autologous Vγ9Vδ2 T lymphocytes amplified ex vivo. However, it is technically simpler to inject directly PAg and IL-2 to the patient (Bennouna J et al. Cancer Immunol Immunother 2008). Several phase I and II clinical trials including as of today about one hundred patients with metastatic renal carcinoma, prostatic carcinoma and follicular lymphoma were performed with this combination therapy. The results of the different trials reveal a very good therapeutic potential of the Vγ9Vδ2 T lymphocytes, but unfortunately a strong toxicity of IL-2.

Together, these therapeutic progress call for a new approach combining PAgs and a safe compound capable of activating the proliferation of Vγ9Vδ2 T lymphocytes different from IL-2 (IL-2Rγc-independent) for a therapeutic approach based on Vγ9Vδ2 T lymphocytes. Different combinations with IL-2Rγc dependant cytokines (IL-4, IL-7, IL-15, IL-21) showed in vitro bioactivities similar to that of IL-2, but also a similar toxicity. Combinations with TLR ligands were also explored by different teams in the world, but revealed a strict IL-2 dependence.

Accordingly there is a need to find an alternative to IL-2 to amplify Vγ9Vδ2 T lymphocytes in vitro or in vivo

The inventor have now surprisingly discovered that the combination of IL-33 and a γ6 T cells activator such as a phosphoantigen is efficient for inducing proliferation of γδ T lymphocytes in vitro from a culture of fresh human PBMC.

The discovery is all the more unexpected since as shown in the example, combinations of two molecules are efficient for inducing and maintaining proliferation of γδ T in vitro and IL-33 is not associated with the IL2 receptor (IL-2Rγc-independent) and therefore able to avoid the IL-2 toxicity.

IL-33 is a cytokine of the IL-1 family which is expressed by the vascular endothelial cells (Cayrol C, Girard J P, Proc Natl Acad Sci USA 2009). This cytokine has a nuclear localization but can be released by stressed or necrotic cells, leading to consider IL-33 as an alarmin. An alarmin is an endogenous signal rapidly released from cells in response to infection or tissue damage (mechanic or induced by chemotherapy or radiotherapy), alarming the immune system by promoting chemoattraction and activation of innate and adaptive immunity (Haraldsen G et al. Trends Immunol 2009). The inventors of the instant invention showed that IL-33 is highly expressed by epithelial cells of tissues in contact with the environment including the skin and gastrointestinal tract, where pathogens, allergens and other environmental agents are frequently encountered. Moreover, they showed that IL-33 is highly expressed by the vascular endothelial cells from HEV (High Endothelial Veinules) which are specialized blood vessels mediating lymphocyte recruitment into lymphoid organs (Moussion C et al. PLoS One 2008).

So far however, no data of literature showed any role of IL-33 on the T VγVδ2 lymphocytes.

### SUMMARY OF THE INVENTION

According to a first aspect, the present invention provides an in vitro or ex vivo method for inducing proliferation of γδ T cells comprising the treatment of said γδ T cells with a combination of IL-33 and a γδ T cells activator.

According to other aspects, the present invention relates to a culture medium or a kit comprising IL-33 and γδ T cells activator (e.g. a phosphoantigen).

According to a further aspect, the present invention provides an ex vivo and/or in vivo method for treating a subject in need of γδ T cell therapy which means for the treatment of infection, autoimmunity, cancer and other proliferative diseases.

According to another aspect, the present invention provides a pharmaceutical composition comprising IL-33 and γδ T cells activator (e.g. a phosphoantigen), and optionally a pharmaceutically acceptable carrier and the use of this pharmaceutical composition in anticancer or anti-infectious therapy.

### DETAILED DESCRIPTION OF THE INVENTION

The present invention arises from the unexpected finding by the inventors that IL-33 can advantageously be used instead of IL-2 in combination with BrHPP, a phosphoantigen, for inducing proliferation of γδ T lymphocytes and allowing further development of the γδ T lymphocytes based therapies. Indeed, the combination of the invention is as efficient for inducing proliferation of γδ T lymphocytes as PAgs/IL-2 combinations and without activating IL2 receptor (IL-2Rγc-independent).

### DEFINITIONS

“Function-conservative variants” are peptides derived from the peptide of the invention in which a given amino acid residue in a protein or enzyme has been changed without altering the overall conformation and function of the polypeptide, including, but not limited to, replacement of an amino acid with one having similar properties (such as, for example, polarity, hydrogen bonding potential, acidic, basic, hydrophobic, aromatic, and the like). Amino acids other than those indicated as conserved may differ in a protein so that the percent protein or amino acid sequence similarity between any two proteins of similar function may vary and may be, for example, from 70% to 99% as determined according to an alignment scheme such as by the Cluster Method, wherein similarity is based on the MEGALIGN algorithm. A “function-conservative variant” also includes a polypeptide which has at least 60% amino acid identity as determined by BLAST or FASTA algorithms, preferably at least 75%, most preferably at least 85%, and even more preferably at least 90%, and which has the same or substantially similar properties or functions as the native or parent protein to which it is compared.

The term “analog”, when used herein in reference to a protein or polypeptide, refers to a peptide that possesses a similar or identical function as the protein or polypeptide but need not necessarily comprise an amino acid sequence that is similar or identical to the amino acid sequence of the protein or polypeptide or a structure that is similar or identical to that of the protein or polypeptide. Preferably, in the context of the present invention, an analog has an amino acid sequence that is at least 80%, more preferably, at least about: 80%, 85%, 90%, 95%, 96%, 97%, 98% or 99%, identical to the amino acid sequence of the protein or polypeptide. In certain preferred embodiments, an analog of a peptide biomarker of the invention has an amino acid sequence that is at least 80% identical or at least 85% identical to the amino acid sequence of the cytokine peptide.

The term “homologous” (or “homology”), as used herein, is synonymous with the term “identity” and refers to the sequence similarity between two polypeptide molecules or between two nucleic acid molecule. When a position in both compared sequences is occupied by the same base or same amino acid residue, the respective molecules are homologous at that position. The percentage of homology between two sequences corresponds to the number of matching or homologous positions shared by the two sequences divided by the number of positions compared and multiplied by 100. Generally, a comparison is made when two sequences are aligned to give maximum homology. Homologous amino acid sequences share identical or similar amino acid sequences. Similar residues are conservative substitutions for, or “allowed point mutations” of, corresponding amino acid residues in a reference sequence. “Conservative substitutions” of a residue in a reference sequence are substitutions that are physically or functionally similar to the corresponding reference residue, e.g., that have a similar size, shape, electric charge, chemical properties, including the ability to form covalent or hydrogen bonds, or the like. Particularly preferred conservative substitutions are those fulfilling the criteria defined for an “accepted point mutation” by Dayhoff et al. (“Atlas of Protein Sequence and Structure”, 1978, Nat. Biomed. Res. Foundation, Washington, D.C., Suppl. 3, 22: 354-352).

In its broadest meaning, the terms “treating” or “treatment” refer to reversing, alleviating, inhibiting the progress of the disorder or condition to which such term applies, or one or more symptoms of such disorder or condition.

The term “patient” refers to any subject (preferably human) afflicted with or susceptible to be afflicted with an infectious disease (bacterial or viral), a cancer or another proliferative disease.

Method for Inducing Proliferation of γδ T Cells Using a γδ T Cell Activator and IL-33

The present invention concerns a novel method for inducing proliferation of γδ T cells wherein γδ T cells are activated in a culture medium containing IL-33 and a γδ T cells activator).

According to a first aspect, the present invention provides an in vitro or ex vivo method for inducing proliferation of γδ T cells comprising the treatment of said γδ T cells with a combination of IL-33 and γδ T cells activator.

In a preferred embodiment, the γδ T cells activator is a phosphoantigen.

In a more preferred embodiment, the phosphoantigen is BrHPP.

The γδ T cells treated with the combination of the invention are preferably Vγ9Vδ2 T cells.

In a specific embodiment, the in vitro or ex vivo method for inducing proliferation of γδ T cells comprises a preliminary step of isolating Peripheral Blood Mononuclear Cells (PBMCs) from blood sample.

In an another embodiment, the present invention provides an in vitro or ex vivo method for inducing IL2 free proliferation of γδ T cells comprising the treatment of said γδ T cells with a combination of IL-33 and γδ T cells activator.

By “IL-33” (also called “Interleukin-33” or “DVS27-related protein” or “IL-1F11” or “interleukin-1 family member 11” or “nuclear factor for high endothelial venules” or “nuclear factor from high endothelial venules”), it refers to the cytokine protein named “IL-33”. The sequence of IL-33 protein and for the different Transcript Variant and/or different biologically active forms of the IL-33 protein which can be used in the present invention may be found at table A:

| SEQ ID | | Nomenclatures used/NCBI ref/ |
|---|---|---|
| number | Sequences | Article and Patent |
| Peptide | MKPKMKYSTN KISTAKWKNT | (IL-33 AA 1-270: full length |
| SEQ ID N° 1 | ASKALCFKLG KSQQKAKEVC | interleukin-33 protein)/(NCBI |
| | PMYFMKLRSG LMIKKEACYF | ref.: NP_254274)/(Cayrol and |
| | RRETTKRPSL KTGRKHKRHL | Girard, PNAS 2009) |
| | VLAACQQQST VECFAFGISG | |
| | VQKYTRALHD SSITGISPIT | |
| | EYLASLSTYN DQSITFALED | |
| | ESYEIYVEDL KKDEKKDKVL | |
| | LSYYESQHPS NESGDGVDGK | |
| | MLMVTLSPTK DFWLHANNKE | |
| | HSVELHKCEK PLPDQAFFVL | |
| | HNMHSNCVSF ECKTDPGVFI | |
| | GVKDNHLALI KVDSSENLCT | |
| | ENILFKLSET | |
| Peptide | AFGISG VQKYTRALHD SSITGISPIT | (IL-33 AA 95-270: 1^{st} natural |
| SEQ ID N° 2 | EYLASLSTYN DQSITFALED | cleavage product of |
| | ESYEIYVEDL KKDEKKDKVL | human IL-33)/ |
| | LSYYESQHPS NESGDGVDGK | NCBI ref.:/Lefrançais et al. |
| | MLMVTLSPTK DFWLHANNKE | PNAS 2012 and WO2012113927) |
| | HSVELHKCEK PLPDQAFFVL | |
| | HNMHSNCVSF ECKTDPGVFI | |
| | GVKDNHLALI KVDSSENLCT | |
| | ENILFKLSET | |
| Peptide | SG VQKYTRALHD SSITGISPIT | (IL-33 AA 99-270) 2^{nd} natural |
| SEQ ID N° 3 | EYLASLSTYN DQSITFALED | cleavage product of |
| | ESYEIYVEDL KKDEKKDKVL | human IL-33)(NCBI ref.:)/ |
| | LSYYESQHPS NESGDGVDGK | (Lefrançais et al. |
| | MLMVTLSPTK DFWLHANNKE | PNAS 2012 and WO2012113927) |
| | HSVELHKCEK PLPDQAFFVL | |
| | HNMHSNCVSF ECKTDPGVFI | |
| | GVKDNHLALI KVDSSENLCT | |
| | ENILFKLSET | |
| Peptide | HD SSITGISPIT EYLASLSTYN | (IL-33 AA 109-270: 3^{rd} natural |
| SEQ ID N° 4 | DQSITFALED ESYEIYVEDL | cleavage product of |
| | KKDEKKDKVL LSYYESQHPS | human IL-33)(NCBI ref.:)/ |
| | NESGDGVDGK MLMVTLSPTK | (Lefrançais et al. |
| | DFWLHANNKE HSVELHKCEK | PNAS 2012 and WO2012113927) |
| | PLPDQAFFVL HNMHSNCVSF | |
| | ECKTDPGVFI GVKDNHLALI | |
| | KVDSSENLCT ENILFKLSET | |
| Peptide | SITGISPIT EYLASLSTYN | (IL-33 AA 112-270: |
| SEQ ID N°: 5 | DQSITFALED ESYEIYVEDL | the artificially truncated |
| | KKDEKKDKVL LSYYESQHPS | form of human IL-33) |
| | NESGDGVDGK MLMVTLSPTK | (NCBI ref.:)/(Schmitz et al. |
| | DFWLHANNKE HSVELHKCEK | Immunity 2005) |
| | PLPDQAFFVL HNMHSNCVSF | |
| | ECKTDPGVFI GVKDNHLALI | |
| | KVDSSENLCT ENILFKLSET | |

IL33 human natural variants for use in the present invention are disclosed in WO2012113927 all of which are herein incorporated by reference

In a preferred embodiment, IL-33 is the first natural cleavage product of human IL-33 (IL-33 AA 95-270: SEQ ID No2)

In another preferred embodiment, IL-33 is the artificially truncated form of human IL-33 (IL-33 AA 112-270: SEQ ID No5).

The cytokine protein IL-33 has been shown to function as a ligand for the IL-1 receptor-related protein ST2 (IL-1R4), a member of the Toll-Like Receptors (TLR)/IL-1 Receptors family (Schmitz J et al. Immunity 2005). ST2 is expressed on Th2 lymphocytes, NKT cells, NK cells and on mast cells, basophils and eosinophils. The interaction IL-33/ST2 leads to an alarming intracellular signal involving the cascade MYD88, MAPK and NF-κB. Accordingly, IL-33 was found to drive production of pro-inflammatory (TNF-alpha, IL-1, IL-6, IFN-gamma) and/or Th2 (IL-4, IL-5, IL-13) cytokines (Pecaric-Petkovic T et al. Blood 2009, Bourgeois E et al. Eur J Immunol 2009) but also to induce chemotaxis of immune cells to the inflammatory site (Komai-Koma M et al. Eur J Immunol 2007).

Broadly, the term “Interleukin-33” refers to the protein IL-33 itself, analogues of the protein, which include polypeptides and proteins which are functionally equivalent to the polypeptide of the invention as well as “function-conservative variants”. In the sense used in the invention, the expression “functionally equivalent” means that the polypeptide in question has at least one of the biological activities of the cytokine of the invention, such as, for example, acting as an activator of the ST2 receptor which leads to an alarming intracellular signal involving the cascade MYD88, MAPK and NF-κB.

The ST2-activating capabilities of the protein of the invention will become evident to the skilled person by implementing a simple test to evaluate the production of pro-inflammatory (TNF-alpha, IL-1, IL-6, IFN-gamma) and/or Th2 (IL-4, IL-5, IL-13) cytokines (Schmitz et al. Immunity 2005, Pecaric-Petkovic T et al. Blood 2009, Bourgeois E et al. Eur J Immunol 2009, Cayrol and Girard PNAS 2009, Lefrancais et al. PNAS 2012) but also to induce chemotaxis of immune cells to the inflammatory site (Komai-Koma M et al. Eur J Immunol 2007).

The term “γδ T cells” (also called “gamma delta T cells” or “γδ T lymphocytes”) represent an important component of the healthy immune system at the interface of the innate and adaptive immunity. γδ T cells are known as non-conventional lymphocytes as they recognize the antigen with their TCR (T cell receptor for the antigen) but without presentation or restriction by molecules of the complex major histocompatibility.

γδ T cells have numerous acknowledged biomarkers known in the art. These include CD3+, CD4−, CD8− and the TCR chain is formed of gamma chain (γ) and delta chain (δ).

Unlike their counterparts αβ T cells, γδ T cells represent only small proportion (<6%) of circulating lymphocytes in the peripheral blood. They are much more prevalent in epithelial tissues and lymphoid organs where they can represent up to 50% of T lymphocytes. However, during various bacterial infections such as tuberculosis, meningitis, or tularemia, and protozoa such as malaria, toxoplasmosis and leishmaniasis, γδ T cells are amplified to levels that can represent the majority of circulating T cells (up to 40% in some individuals.

γδ T cells according to the present invention are primate γδ T cells, most preferably human γδ T cells.

Detection of γδ T cell proliferation can be performed by standard methods. One specific method for detecting γδ T cell proliferation in vitro is described in Example 1.

By “Vγ9Vδ2 T cells” (also called “Vγ9Vδ2 T lymphocytes”), is meant a subgroup of γδ T cells present only in primates (human and nonhuman) with a TCR of type Vγ9Vδ2. The antigens selectively recognized by human Vγ9Vδ2 T lymphocytes are non peptidic antigens called phosphoantigens (PAgs).

By their production of pro-inflammatory cytokine and their cytotoxicity induced by their activation, Vγ9Vδ2 T cells are very important actors of the antitumor immunity. They have indeed a high cytolitic potential in vitro against numerous cancer cell types as established cancer cell lines or cells from cancer patients.

“Vγ9Vδ2 T cells” may be isolated from PBMCs by any suitable method known in the art. Examples of such methods are set out in the example section.

For example, the initial cell preparation consists of PBMCs from blood from either fresh or frozen cytapheresis. The cells are expanded for two weeks in a closed system, with sequential addition of defined dosage IL-33 to the culture medium after a unique PAgs stimulation. The manufacturing process is much simpler than most current cellular therapy approaches using conventional CD8+ T cell lines or clones: there is no final separation or purification step nor use of feeder cells; the specific TCR-mediated signal provided by PAgs is sufficient to trigger the IL-33-dependent expansion of the Vγ9Vδ2 subset, which becomes dominant in the culture. Several doses of the γδ cellular product can be manufactured from one frozen cytapheresis.

Typically, 100 million frozen PBMCs from cytapheresis yield 2 to 5 billions cells with the classical method of amplification with a γδ T cell specific stimulus (e.g. phosphoantigen) and IL-2.

A Vγ9Vδ2 γδ T cell must preferably display cytotoxic function against tumor cells. The demonstration of cytotoxic function may be determined by any suitable method known in the art. In particular, examples of such tests are set out in the example section. Specifically, the tests embodied in example and FIG. 1 are regarded as standards in vitro tests for the assessment of Vγ9Vδ2 γδ T cell function.

The term “γδ T cell activator” designates a molecule, preferably artificially produced, which activates γδ T lymphocytes. It consists more preferably of a ligand for the γδ T lymphocyte's TCR and other receptor expressed on γδ T cell like activator receptor of NK cells (NKG2D . . . ). The activator may be of various natures, such as a peptide, a lipid or is a small chemical molecule (e.g. phosphoantigen), It also may be a ligand, or a fragment or derivative thereof, or an antibody having substantially the same specificity for the γδ T lymphocyte's TCR and other receptor of γδ T cell like activator receptor of NK cells (NKG2D . . . ).

In particular embodiment the γδ T cell activator is a γδ T cell-specific activator which activates only the γδ T lymphocytes among all lymphocytes (For instance with a EC50 less or equal at 24 nM)

The γδ T cell activator is preferably purified from cells or otherwise artificially produced (e.g., by chemical synthesis, or by microbiological process).

By “Phosphoantigens” (also called “PAgs”) refers to nonpeptide phosphate compound typically mono- and pyro-phosphates of linear C5 isoprenoids with bioactivity of γδ T cell activator. All phosphoantigens owe their antigen bioactivity to their phosphate moiety, which bioactivity is abrogated by phosphatases.

A phosphoantigen that is a γδ T cell activator preferably increases the biological activity or causes the proliferation of γδ T cells and preferably increases the activation of γδ T cells, particularly the cytokine secretion from γδ T cells or the cytolytic activity of γδ T cells, with or without further stimulating the proliferation or expansion of γδ T cells in association with interleukin like IL-2.

Accordingly, the γδ T cell activator is added to the cell culture or administered in an amount and under conditions sufficient to increase the activity of γδ T cells in a subject, preferably in an amount and under conditions sufficient to increase cytokine secretion by γδ T cells and/or to increase the cytolytic activity of γδ T cells. Cytokine secretion and cytolytic activity can be assessed by any appropriate in vitro assays.

Cytokine secretion can be determined according to the methods described in Espinosa et al. (J Biol. Chem., 2001, Vol. 276, Issue 21, 18337-18344), describing measurement of TNF-α release in a bioassay using TNF-α-sensitive cells.

The phosphoantigens for use in the invention may be obtained by purification from micro-organisms and plants, or by any synthetic method or by microbiological process, well known to the skilled person.

Natural PAg such as isopentenyl pyrophosphate (IPP) described in U.S. Pat. No. 5,639,653, dimethylallyl pyrophosphate (DMAPP), 3-formyl-butyl-pyrophosphate, and 4-hydroxy-3-dimethylallyl pyrophosphate (HDMAPP) which is synonymous to (E)-4-hydroxy-3-methyl-but-2-enyl pyrophosphate (HMBPP). These molecules have been identified in several micro-organisms and plants. Literature equally refers to HDMAPP or HMBPP for the same above depicted molecule of (E)-4-hydroxy-3-methyl-but-2-enyl pyrophosphate structure.

Other phosphoantigens for use in the present invention with significant γδ T cell activating activity are disclosed in WO 95/20673, WO 2004/050096, WO2007/057440, WO2007039635 and Belmant et al (Drug Discovery Today: Therapeutic Strategies 2006 (3), 17-23) all of which are herein incorporated by reference. Still other PAgs are alkylamines (such as ethylamine, iso-propyulamine, n propylamine, n-butylamine and iso-butylamine, for instance). Isobutyl amine and 3-aminopropyl phosphonic acid are obtained from Aldrich (Chicago, Ill.).

Preferably, the phosphoantigen is a compound of formula (I):

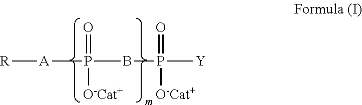

wherein Cat+ represents one (or several, identical or different) organic or mineral cation(s) (including proton);

m is an integer from 0 to 3;
B is O, NH, or any group able to be hydrolyzed;

Y is O⁻Cat+, a C₁-C₃ alkyl group, a group -A-R, or a radical selected from the group consisting of a nucleoside, an oligonucleotide, a nucleic acid, an amino acid, a peptide, a protein, a monosaccharide, an oligosaccharide, a polysaccharide, a fatty acid, a simple lipid, a complex lipid, a folic acid, a tetrahydrofolic acid, a phosphoric acid, an inositol, a vitamin, a co-enzyme, a flavonoid, an aldehyde, an epoxyde and a halohydrin;

A is O, NH, CHF, CF₂ or CH₂;

R is a linear, branched, or cyclic, aromatic or not, saturated or unsaturated, C₁-C₅₀ hydrocarbon group, optionally interrupted by at least one heteroatom, wherein said hydrocarbon group comprises an alkyl, an alkylenyl, or an alkynyl, preferably an alkyl or an alkylene, which can be substituted by one or several substituents selected from the group consisting of: an alkyl, an alkylenyl, an alkynyl, an epoxyalkyl, an aryl, an heterocycle, an alkoxy, an acyl, an alcohol, a carboxylic group (—COOH), an ester, an amine, an amino group (—NH₂), an amide (—CONH₂), an imine, a nitrile, an hydroxyl (—OH), a aldehyde group (—CHO), an halogen, an halogenoalkyl, a thiol (—SH), a thioalkyl, a sulfone, a sulfoxide, and a combination thereof.

In a more preferred embodiment, the phosphoantigen is a compound of formula (II):

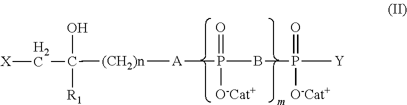

in which X is an halogen (preferably selected from I, Br and Cl), B is O or NH, m is an integer from 1 to 3, R1 is a methyl or ethyl group, Cat+ represents one (or several, identical or different) organic or mineral cation(s) (including the proton), and n is an integer from 2 to 20, A is O, NH, CHF, CF₂ or CH₂, and Y is O⁻Cat+.

For X=Br, R1=methyl, A=O, B=O: the γδ T cell activator is named BrHPP

For X=Br, R1=methyl, A=O, B=CHF, CF₂ or CH₂: the γδ T cell activator is named C-BrHPP

For X=Br, R1=methyl, A=O, B=NH: the γδ T cell activator is named N-BrHPP

In particular, the γδ T cell activator can be BrHPP, C-BrHPP or N-BrHPP.

In another more preferred embodiment, the phosphoantigen is a compound of formula (III):

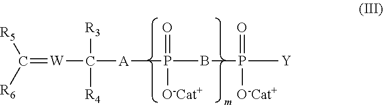

in which R₃, R₄, and R₅, identical or different, are a hydrogen or (C₁-C₃)alkyl group, W is —CH— or —N—, R₆ is an (C₂-C₃)acyl, an aldehyde, an (C₁-C₃)alcohol, or an (C₂-C₃)ester, Cat+ represents one (or several, identical or different) organic or mineral cation(s) (including the proton), B is O or NH, m is an integer from 1 to 3, A is O, NH, CHF, CF₂ or CH₂, and Y is O⁻Cat+.

In particular, the γδ T cell activator can be HDMAPP, C-HDMAPP or N-HDMAPP, which is synonymous to HMBPP, C-HMBPP or N-HMBPP.

For R6=CH₂OH, R5=methyl, W=CH, R3=R4=H, A=O, B=O: the γδ T cell activator is equally named HDMAPP or HMBPP,

For R6=CH₂OH, R5=methyl, W=CH, R3=R4=H, A=CHF, CF₂ or CH₂, B=O: the γδ T cell activator is equally named C-HDMAPP or C-HMBPP,

For R6=CH₂OH, R5=methyl, W=CH, R3=R4=H, A=NH, B=O: the γδ T cell activator is equally named N-HDMAPP or N-HMBPP,

Preferably, the γδ T cell activator can be an aminophosphonate of formula IV:

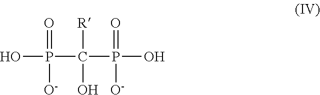

with R′ being a linear, branched, or cyclic, aromatic or not, saturated or unsaturated, C₁-C₅₀ hydrocarbon group, wherein said hydrocarbon group comprises an alkyl, an alkylenyl, or an alkynyl, preferably an alkyl or an alkylene, which is substituted by one or several substituents selected from the group consisting of: an amine, an amino group (—NH₂), an amide (—CONH₂), an imine, and a combination thereof.

In a preferred embodiment, R′ of formula IV is a linear, branched, or cyclic, aromatic or not, saturated or unsaturated, C₁-C₁₀ hydrocarbon group, which is substituted by an amine, an amino group, a pyridine group, a pyrimidine group, a pyrrole group, an imidazole group, a pyrazole group, a triazole group.

In a still more preferred embodiment, R′ of formula IV is selected from the group consisting of:

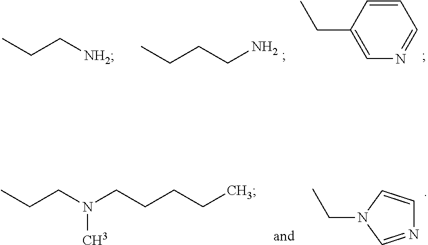

In particular, the γδ T cell activator can be selected from the group consisting of pamidronate, alendronate, ibandronate, risedronate and zoledronate.

Another aspect of the invention is the in vitro and/or ex-vivo use of IL-33 and a γδ T cells activator for inducing proliferation of γδ T cells.

In a preferred embodiment the γδ T cells activator is a phosphoantigen

In the preferred embodiment the phosphoantigen is BrHPP

In a preferred embodiment the γδ Tcell treated with the combination of the invention is Vγ9Vδ2 T cells.

In the most preferred embodiment, inducing proliferation of γδ T cells comprises inducing proliferation of Vγ9Vδ2 T cells with IL 33, and BrHPP.

The dose used for IL-33 is between 1 and 1000 ng/ml, preferably between 10 ng/ml and 1000 ng/ml, most preferably 500 ng/ml.

The dose used for BrHPP is between 1 and 1000 nM, preferably between 10 nM and 200 nM, most preferably 100 nM.

The Vγ9Vδ2 T cells expanded by the method of the invention may be cultured between four and twenty one preferably between four and fifty days and most preferably during fourteen days.

Ex vivo stimulation allows the generation of critical numbers of Vγ9Vδ2 T cells for therapeutic purposes. The BrHPP-stimulated γδ T cells may be obtained by a 2-week manufacturing process.

It should be noted that the major advantage of the present invention is inducing specific proliferation of γδ T cells by an IL2 independent process. This is in contrast with the prior art where the major effect shown to date is the expansion of Vγ9Vδ2 T cells but with high toxicity of IL2.

The opportunity to actively inducing proliferation of Vγ9Vδ2 T cells in an IL2 independent way is a significant advantage of the present invention and represents a safe alternative to IL2 with a higher rate of proliferation (see example 2 and FIG. 1A).

Culture Medium, Kit and Method for Expending Treg Cells

The present invention also relates to a culture medium comprising a γδ T cell activator and IL 33.

The culture medium of the present invention is suitable for inducing proliferation of γδ T cells for activating their therapeutic function.

The term “culture medium” as used herein refers to a liquid medium suitable for the in vitro culture of γδ T cell, preferably manufactured at clinical grade. Typically, the culture medium of the invention contains:
a source of carbon as energy substrate, such as glucose, galactose or sodium pyruvate;
essential amino-acids;
vitamins, such as biotin, folic acid, B12 . . . ;
at least a purine and a pyrimidine as nucleic acid precursors;
inorganic salts;

The culture medium may also contain pH buffers in order to maintain the pH of the medium at a value suitable for cell growth.

The culture medium of the invention may be based on a commercially available medium such as RPMI 1640 supplemented with foetal calf serum.

Another aspect of the invention relates to an in vitro method for inducing proliferation of γδ T cells wherein said method comprises the step of culturing of γδ T cells with the culture medium as described above.

The step of culturing of γδ T cells with the culture medium of the invention shall be carried out for the necessary time required for the production of functional of γδ T cells. Typically, the culture of γδ T cells with the medium of the invention shall be carried out for at least 4 days, preferably at least 5 days, preferably at least 10 days, even more preferably at least 14 days.

If necessary, the culture medium of the invention can be renewed, partly or totally, at regular intervals. Typically, the culture medium of the invention is regularly replaced with fresh culture medium of the invention for example every 3 day, for the whole culture.

Another aspect of the invention is a kit comprising: (i) γδ T cells activator and (ii) IL-33.

In a preferred embodiment the γδ T cells activator is a phosphoantigen

In the preferred embodiment the phosphoantigen is BrHPP

Method of Treatment and Pharmaceutical Compositions

A further aspect of the present invention provides an ex vivo and/or in vivo method for treating a subject in need of γδ T cell therapy namely for the treatment of infection autoimmunity, cancer, as well as other proliferative diseases.

Thus, a further aspect of the invention relates to an ex vivo method of treating a subject in need of γδ T cell therapy comprising
(i) removing a blood sample comprising γδ T cells from a subject
(ii) isolating PBMC from blood sample
(iii) treating PBMC with (i) a γδ T cells activator and (ii) IL-33 in order to obtain between 1 to 5 billions γδ T cells
(iv) reintroducing the PBMC culture enriched in γδ T cells so obtained (amplified) into said subject

Typically, 100 million frozen PBMCs from cytapheresis yield 2 to 5 billions cells.

The Phosphoantigen-stimulated γδ T cells have been previously used in a Phase I clinical trial in metastatic Renal Cell Carcinoma (mRCC). The trial was performed with a second dose level of 4 billions cells after achieving correct tolerance of the first 1 billion cell dose.

In a preferred embodiment the γδ T cells treated are Vγ9Vδ2 T cells.

In a preferred embodiment the γδ T cells activator is a phosphoantigen

In the preferred embodiment the phosphoantigen is BrHPP

Another aspect of the invention relates to an in vivo method for treating or preventing infection, autoimmunity, cancer, and other proliferative diseases, comprising administering to a subject in need thereof a therapeutically effective amount of (i) a γδ T cells activator and (ii) IL-33 as described above.

In this aspect, the present invention relates to methods for the treatment of infection, autoimmunity, cancer, and other proliferative diseases, and more preferably a solid tumor, particularly a solid tumor having metastases, where a γδ T cell activator, especially a phosphoantigen, especially a γδ T cell activator according to formulas I to IV, especially γδ T cell activator selected from the group consisting of BrHPP, and HDMAPP, is administered with IL-33 in an amount and under conditions sufficient to stimulate the expansion of the γδ T cell population in a subject, particularly to reach 30-90% of total circulating lymphocytes, typically 40-90%, more preferably from 50-90%. In typical embodiments, the invention allows the selective expansion of γδ T cells in a subject, to reach 60-90% of total circulating lymphocytes, preferably 70-90%, more preferably from 80-90%. Percentage of total circulating lymphocytes can be determined according to methods known in the art. A preferred method for determining the percentage of γδ T cells in total circulating lymphocytes is by flow cytometry.

The method and combination for uses according to the invention is used in a patient in need of γδ T cell therapy namely for the treatment of infection, autoimmunity, cancer, and other proliferative diseases.

A variety of cancers and other proliferative diseases including, but not limited to the following can be treated using the methods and compositions of the invention:
carcinoma, including that of the bladder, breast, colon, kidney, liver, lung, ovary, pancreas, stomach, cervix, thyroid and skin, including squamous cell carcinoma;
tumors of mesenchymal origin, including fibrosarcoma and rhabdomyoscarcoma;
other tumors, including melanoma, seminoma, teratocarcinoma, neuroblastoma and glioma;
tumors of the central and peripheral nervous system, including astrocytoma, neuroblastoma, glioma, and schwannomas;
tumors of mesenchymal origin, including fibrosarcoma, rhabdomyoscaroma, and osteosarcoma; and
other tumors, including melanoma, xeroderma pigmentosum, keratoacarcinoma, 20 seminoma, thyroid follicular cancer and teratocarcinoma.
leukemias such as, but not limited to, acute leukemia, acute lymphocytic leukemia, acute myelocytic leukemias such as myeloblastic, promyelocytic, myelomonocytic, monocytic, erythroleukemia leukemias and myelodysplastic syndrome, chronic leukemias such as but not limited to, chronic myelocytic (granulocytic) leukemia, chronic lymphocytic leukemia, hairy cell leukemia; polycythemia vera;
lymphomas such as, but not limited to, Hodgkin's disease, non-Hodgkin's disease; multiple myelomas such as, but not limited to, smoldering multiple myeloma, nonsecretory myeloma, osteosclerotic myeloma, plasma cell leukemia, solitary plasmacytoma and extramedullary plasmacytoma.

Where hereinbefore and subsequently a tumor, a tumor disease, a carcinoma or a cancer are mentioned, metastasis in the original organ or tissue and/or in any other location are implicitly meant alternatively or in addition, whatever the location of the tumor and/or metastasis is.

In one embodiment, the cancer is selected from the group consisting of renal carcinoma, prostatic carcinoma and follicular lymphoma.

A variety of infectious diseases including but not limited to the following can be treated using the methods and compositions of the invention: viral infection, bacterial infection, parasitic (protozoan) infection, and fungal infection,

A variety of autoimmune diseases including but not limited to the following can be treated using the methods and compositions of the invention to: Ankylosing Spondylitis, Crohns Disease (one of two types of idiopathic inflammatory bowel disease “IBD”) Dermatomyositis, Diabetes mellitus type 1, Lupus erythematosus, Multiple Sclerosis, Psoriasis, Psoriatic Arthritis, Rheumatoid arthritis, Vasculitis

According to another aspect, the present invention provides a pharmaceutical composition comprising (i) IL-33 and (ii) a γδ T cells activator (e.g. a phosphoantigen) and optionally a pharmaceutically acceptable carrier and the use of this pharmaceutical composition in therapy of infection, autoimmunity cancer, as well as other proliferative diseases.

The therapeutic ingredients of the invention may be combined with pharmaceutically acceptable excipients, and optionally sustained-release matrices, such as biodegradable polymers, to form therapeutic compositions.

“Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

The form of the pharmaceutical compositions, the route of administration, the dosage and the regimen naturally depend upon the condition to be treated, the severity of the illness, the age, weight, and sex of the patient, etc.

The pharmaceutical compositions of the invention can be formulated for a topical, oral, intranasal, parenteral, intraocular, intravenous, intramuscular or subcutaneous administration and the like.

In another aspect, the invention provides a combination of: (i) a γδ T cells activator and (ii) IL-33 as described above, which may be used for the preparation of a pharmaceutical composition for the treatment of infection, autoimmunity, cancer, as well as other proliferative diseases.

Compounds of the invention may be administered in the form of a pharmaceutical composition, as defined below.

By a “therapeutically effective amount” is meant a sufficient amount of compound to treat and/or to prevent, reduce and/or alleviate one or more of the symptoms of cancer and infectious disease.

### Administration of the Combination Treatment

It will be understood that the total daily usage of the compounds and compositions of the present invention will be decided by the attending physician within the scope of sound medical judgment. The specific therapeutically effective dose level for any particular patient will depend upon a variety of factors including the disorder being treated and the severity of the disorder; activity of the specific compound employed; the specific composition employed, the age, body weight, general health, sex and diet of the patient; the time of administration, route of administration, and rate of excretion of the specific compound employed; the duration of the treatment; drugs used in combination or coincidental with the specific compound employed; and like factors well known in the medical arts. For example, it is well known within the skill of the art to start doses of the compound at levels lower than those required to achieve the desired therapeutic effect and to gradually increase the dosage until the desired effect is achieved.

In one embodiment, the cytokine IL-33 and the γδ T cell activator (or the activated γδ T cells) are administered into the subject simultaneously or sequentially. In a first embodiment, the γδ T cell activator (or the activated γδ T cells) is administered to the subject before the cytokine IL-33. In a second embodiment, the cytokine IL-33 is administered to the subject before the γδ T cell activator (or the activated γδ T cells). The γδ T cell activator (or the activated γδ T cells) and the cytokine IL-33 are administered so that the combined effect can be obtained.

In another aspect, the invention provides a combination of a γδ Tcell activator and IL-33 for the treatment of infection, autoimmunity, cancer, as well as other proliferative diseases, wherein the γδ Tcell activator and IL-33 are administrated simultaneously or sequentially.

The γδ T cell activator may be administered only as a single dose to the individual. In another aspect, the γδ T cell activator is administered in multiple doses, the administration of successive doses of the γδ T cell activator being separated by at least 2, 3 or 4 or more weeks. Generally, the γδ T cell rate (number of γδ T cells), is allowed to return to substantially the basal rate prior to a second administration of the compound. At least about one week, but more preferably at least about two weeks, or up to eight weeks are required for a patient's γδ T cell rate to return to substantially the basal rate. For example, the γδ T cell activator can be administered only as a single dose to the individual, which will usually mean that the γδ T cell activator is administered no more than once a month or once every 2, 3 or 6 months.

In a preferred aspect, the γδ T cell activator may increase the biological activity of γδ T cells, preferably increasing the activation of γδ T cells, particularly increasing cytokine secretion from γδ T cells or increasing the cytolytic activity of γδ T cells, with stimulating the expansion of γδ T cells with IL33. Thus in one aspect, the present invention relates to methods for the treatment of infection, autoimmunity, cancer, as well as other proliferative diseases, and more preferably a solid tumor, particularly a solid tumor having metastases, where a γδ T cell activator, especially a phosphoantigen, especially a γδ T cell activator according to formulas I to IV, especially γδ T cell activator selected from the group consisting of BrHPP, and HDMAPP, is administered with IL-33 in an amount and under conditions sufficient to increase cytokine secretion by γδ T cells and/or to increase the cytolytic activity of γδ T cells. In typical embodiments, a γδ T cell activator allows the cytokine secretion by γδ T cells to be increased at least 2, 3, 4, 10, 50, 100-fold, as determined in vitro.

Preferably, dosage (single administration) of a compound of formula I for treatment is between about 1 mg/kg and about 1.2 g/kg.

It will be appreciated that the above dosages related to a group of compounds, and that each particular compound may vary in optimal doses, as further described herein for exemplary compounds. Nevertheless, compounds are preferably administered in a dose sufficient to significantly increase the biological activity of γδ T cells or to significantly increase the γδ T cell population in a subject. Said dose is preferably administered to the human by intravenous (i.v.) administration during 2 to 180 min, preferably 2 to 120 min, more preferably during about 5 to about 60 min, or most preferably during about 30 min or during about 60 min.

In preferred exemplary compounds, a compound of formula II is administered in a dosage (single administration) between about 0.1 mg/kg and about 1.2 g/kg, preferably between about 10 mg/kg and about 1.2 g/kg, more preferably between about 5 mg/kg and about 100 mg/kg, even more preferably between about 5 mg/kg and 60 mg/kg. Most preferably, dosage (single administration) for three-weekly or four-weekly treatment (treatment every three weeks or every third week) is between about 0.1 mg/kg and about 1.2 g/kg, preferably between about 10 mg/kg and about 1.2 g/kg, more preferably between about 5 mg/kg and about 100 mg/kg, even more preferably between about 5 mg/kg and 60 mg/kg. This dose is preferably administered to the human by intravenous (i.v.) administration during 2 to 180 min, preferably 2 to 120 min, more preferably during about 5 to about 60 min, or most preferably during about 30 min or during about 60 min.

An IL-33 cytokine having γδ T cell proliferation inducing activity, most preferably the IL-33 polypeptide, is administered at low doses, typically over a period of time comprised between 1 and 10 days. The γδ T cell activator is preferably administered in a single dose, and typically at the beginning of the γδ T cell activator treatment.

In preferred aspects, a IL-33 cytokine, is administered daily for up to about 10 days, preferably for a period of between about 3 and 10 days, or most preferably for about 5 days. Preferably, the administration of the cytokine begins on the same day (e.g. within 24 hours of) as the administration of the γδ T cell activator. It will be appreciated that the cytokine can be administered in any suitable scheme within said regimen of between about 3 and 10 days. When the cytokine is administered for about 7 to about 14 days, a 4-weekly treatment cycle is preferred. When the first component is administered for about 4 days, a 3-weekly day treatment cycle is preferred

The IL-33 polypeptide is preferably administered at low doses, i.e. at doses that are sufficient to target in vivo cells that express the high affinity receptor for IL-33, defined as IL-1 receptor-related protein ST2 (IL-1R4). Practically, in human, such doses have been experimentally defined (in clinical trials with IL2) as being comprised between 0.2 and 2 million units per square meters, when injected subcutaneously. The IL-33 polypeptide is preferably administered by injection of between 0.1 and 3 million units (MU) per day, over a period of 1 to 10 days. Preferably, daily doses of between 0.2 and 2 MU per day, even more preferably between 0.2 and 1.5 MU, further preferably between 0.2 and 1 MU, are being administered. The daily dose may be administered as a single injection or in several times, typically in two equal injections. The IL-33 treatment is preferably maintained over between 1 and 9 days, even more preferably during 3 to 7 days. Optimum effect seems to be achieved after 5 days treatment.

The therapeutic agents of the invention may further be combined with other active ingredients, for example chemotherapeutics, anti-metastatic or anti-cancer or anti-proliferative agents.

In one specific embodiment, such compound may be combined with compounds drugs appropriate for cancer therapy, for example, drugs selected from the group consisting of: immunotherapeutic drugs (Imids), therapeutic monoclonal antibodies, and biological therapeutics.

In another aspect, the invention provides (i) a γδ T cells activator and (ii) IL-33 as described above, which may be used in combination with interferon for the treatment of infectious disease, in particularly for the treatment of CMV infection, and more particularly for the treatment of hepatitis B.

The invention will be further illustrated by the following figures and examples. However, these examples and figures should not be interpreted in any way as limiting the scope of the present invention.

### FIGURES

FIG. 1: IL-33 increases Vγ9Vδ2 T lymphocytes proliferation.

A. CFSE dilution of gated Vγ9Vδ2 T lymphocytes after a six days culture of PBMC activated or not with BrHPP (100 nM), in the presence or not of IL-2 (10 UI/ml) and IL-33 (1, 10, 1000 ng/ml); ctrl: control without IL-33. Representative experiment of one donor among seven.

B. Absolute number of Vγ9Vδ2 T lymphocytes after a six days culture of PBMC activated with BrHPP (100 nM) and in the presence or not of IL-33 (1000 ng/ml) or IL-2 (10 UI/ml) (mean±SD from 7 independent experiments).

FIG. 2:

A. Expression of annexine V and PI by γδT cells after one day or three days of culture with or without IL-33.

B. Percentage of living γδ T cells and living PBMC after one or three days of culture with or without IL-33 (mean of 2 independent donors).

### EXAMPLE 1

### Methods

PBMC and PBL Preparation

Fresh blood samples were collected from healthy donors, and PBMC were prepared on a Ficoll-Paque density gradient (Amersham Biosciences AB, Upssala, Sweden) by centrifugation (800 g, 30 min at room temperature).

PBL (peripheral blood lymphocytes) were prepared from monocytes depletion on PBMC by magnetically activated cell sorting using the CD14 MicroBead Kit (Miltenyi Biotec, Auburn, Calif.) accordingly to the manufacturer's instructions.

Cell Cultures

Cell cultures were performed in complete medium: RPMI 1640 supplemented by penicillin 100 UI/ml, streptomycin 100 μg/ml, L-glutamin 2 mM, sodium pyruvate 1 mM and 10% FCS.

Proliferation Assays

PBMC or PBL were labeled with 0.125 μM CFSE (Invitrogen, France) for 8 min at 37° C. and cultured in 96-well plates (3.10⁵ cells per well) in 200 μl of complete medium with or without BrHPP [100 nM], IL-33 [0-1000 ng/ml] and with or without rhIL-2 [10 UI/ml] (Sanofi Aventis, France). After six days in culture CFSE dilution was evaluated in γδ T cells (CD3⁺ γ9 TCR cells) by flow cytometry.

Cell amplification was evaluated with a cell counter based on the Propidium Iodure detection.

The IL-33 forms used in this example was the natural cleavage product of human IL-33 (IL-33 aa 95-270) and the truncated form of human IL-33 (IL-33 aa 112-270).

Antibodies Staining

Anti-TCR γ9-APC and anti-CD3-Pacific blue were used to select Vγ9Vδ2 T lymphocytes by flow cytometry.

Statistical Analysis

Significant differences were assessed with Student's t-test by using the SigmaStat software (Systat Software Inc., San Jose, Calif.).

### EXAMPLE 2

### Results

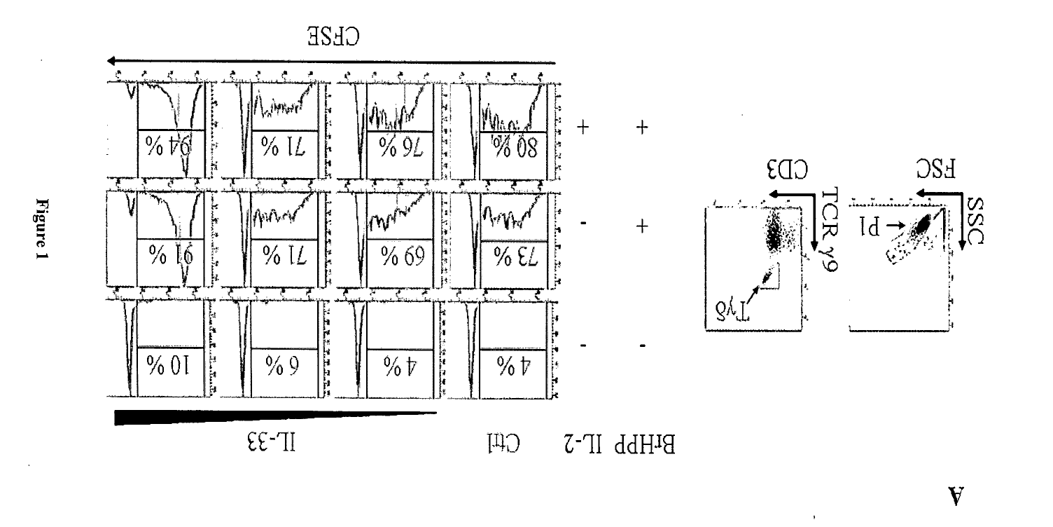

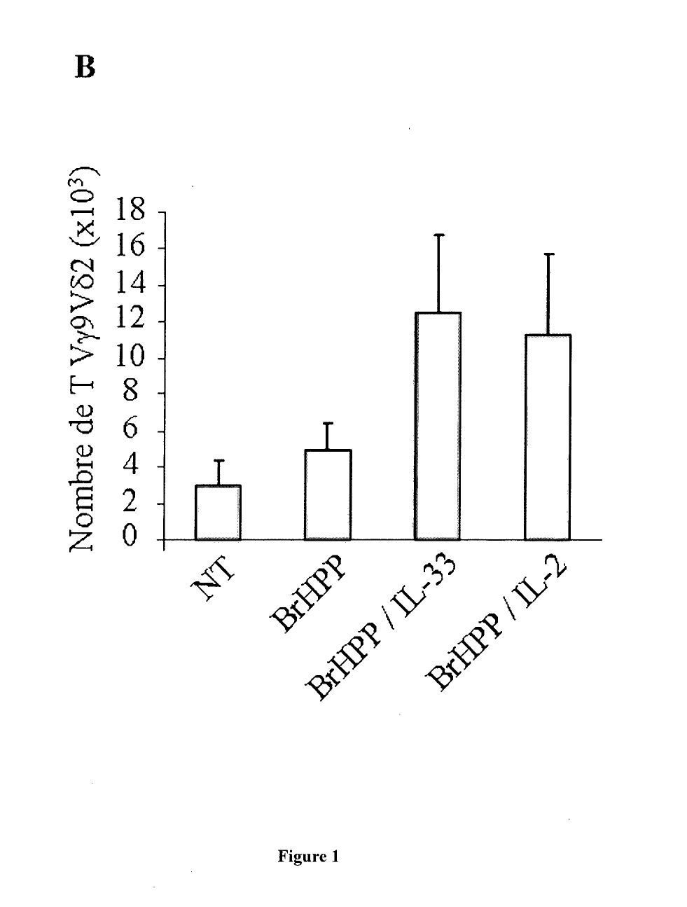

We studied the effect of IL-33 in combination with the specific phosphoantigen, BrHPP, on the proliferation of human Vγ9Vδ2 T lymphocytes. In this purpose, fresh PBMC were stained with CFSE and cultured with or without BrHPP, IL-33 and IL-2. After six days in culture, the proliferation of Vγ9Vδ2 T lymphocytes was analyzed by the reduction of the CFSE fluorescence intensity on the Vγ9Vδ2 T lymphocytes gated by flow cytometry. We showed in FIG. 1A that without specific BrHPP-activation, Vγ9Vδ2 T lymphocytes are not able to proliferate with or without IL-33. The addition of 100 nM BrHPP in the culture induces the proliferation of Vγ9Vδ2 T lymphocytes. The combination of BrHPP and 1000 ng/ml of IL-33 amplifies significantly this proliferation more than the combination of BrHPP and IL-2. Regarding to the absolute number of Vγ9Vδ2 T lymphocytes obtained after six days in the culture, the combination of IL-33 with BrHPP increases this number in a similar manner as the combination of BrHPP and IL-2 (FIG. 1B).

Discussion

Today, the only tool to amplify the Vγ9Vδ2 T lymphocytes in vitro or in vivo is the use of IL-2 combined to PAgs. Antitumor clinical trials based on Vγ9Vδ2 T lymphocytes consist today either of an injection of high quantities of Vγ9Vδ2 T lymphocytes obtained by an in vitro culture with PAgs and IL-2 or of a direct injection of the two molecules allowing the in vivo amplification of Vγ9Vδ2 T lymphocytes. The first protocol has unfortunately limitations due to the deficient antitumor functionality after injection in the patient of the in vitro generated lymphocytes. The direct injection of PAgs and IL-2 show a good antitumor efficacy thanks to the cytotoxicity of the Vγ9Vδ2 T lymphocytes against cancer cells. However, the high toxicity of IL-2 represents an important limitation of this therapy despite the benefic effects. Actually, the benefit (antitumor cytotoxivity of Vγ9Vδ2 T lymphocytes)/risk (toxicity of IL-2) ratio is not in favor of this therapy.

The present invention seeks to overcome these problems and opens thus real perspectives for the use of IL-33 in antitumor therapies based on Vγ9Vδ2 T lymphocytes. Actually, the capacity of IL-33 in combination with PAgs to amplify the Vγ9Vδ2 T lymphocytes could have important applications. IL-33 could replace IL-2 in therapies based on the injection of PAgs/IL-2 combinations to increase the benefit/risk ratio as IL-33 is less toxic than IL-2.

### EXAMPLE 3

### Tests of IL-33 Toxicity In Vitro

Materials and Methods

PBMC freshly isolated from blood sample from healthy donors were cultured for 6 days in complete RMPI supplemented by 10% FCS in the presence or not of IL-33 (0, 100, 500, 1000, 10000 ng/ml).

At days 1 and 3, PBMC were tested for their viability with a staining with annexin V and propidium iodure (PI) following by the analysis by flow cytometry.

Results

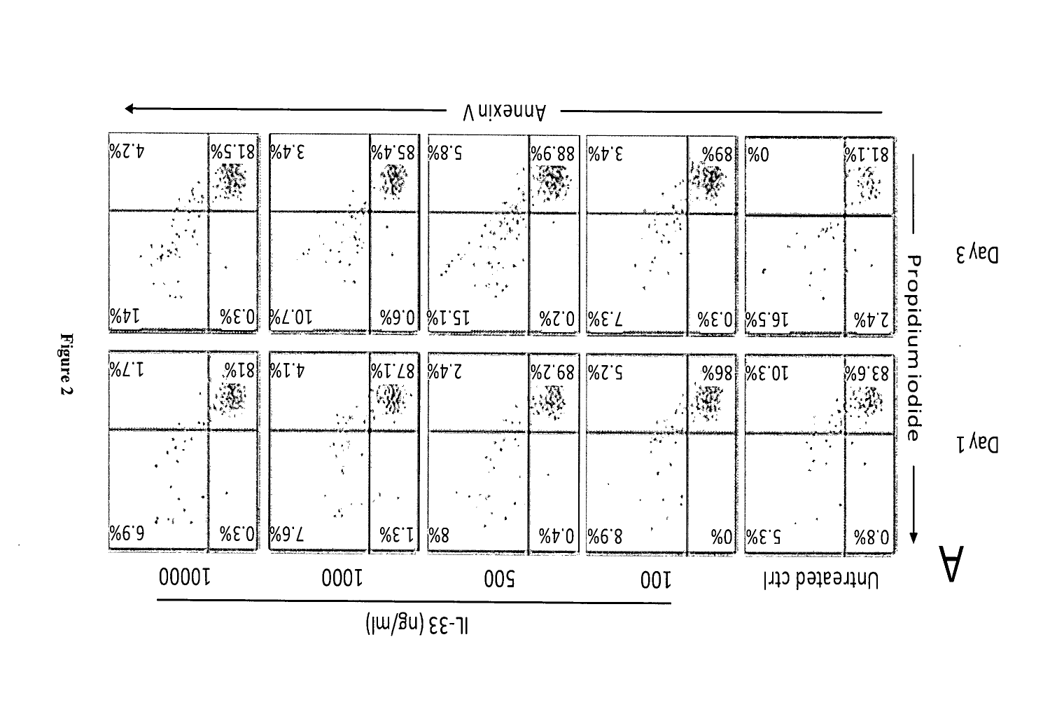

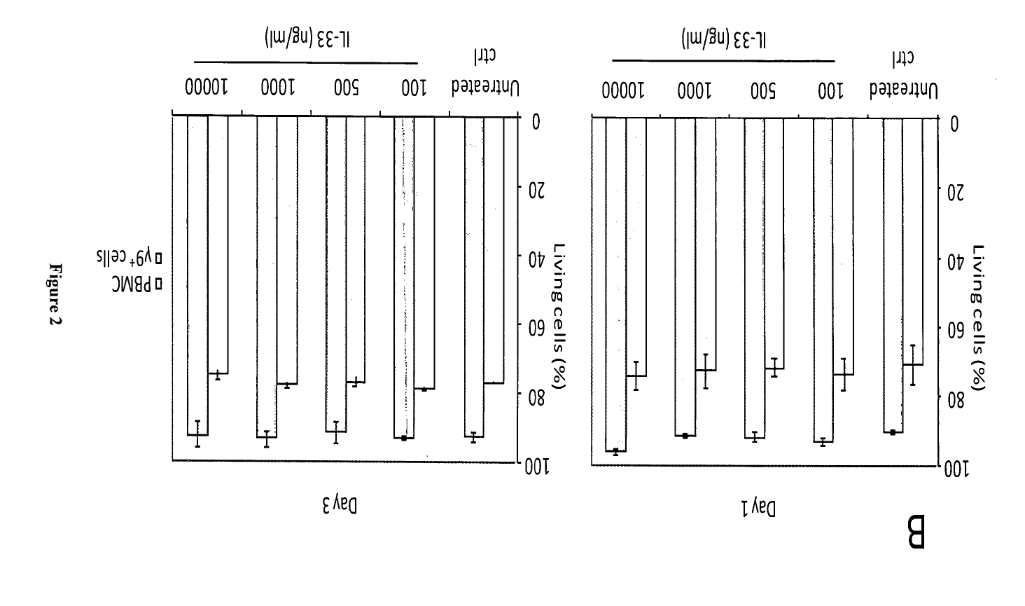

FIG. 2A shows the viability of T cells gated through the staining with annexin V and PI. The percentage of cells negative for annexin V and PI represents the living cells. After one or three days of culture with or without IL-33, over 95% of T cells were alive.

FIG. 2B shows that the percentage of living PBMC or living T cells is constant regardless the IL-33 concentration.

Thus, we demonstrated that IL-33 is not toxic for human PBMC and particularly for human γδ T cells cultured in vitro even at a high dose of IL-33.

### REFERENCES

Throughout this application, various references describe the state of the art to which this invention pertains. The disclosures of these references are hereby incorporated by reference into the present disclosure.
Cayrol C & Girard J P (2009) The IL-1-like cytokine IL-33 is inactivated after maturation by caspase-1. Proc Natl Acad Sci USA 106:9021-9026.
Schmitz J, et al. (2005) IL-33, an interleukin-1-like cytokine that signals via the IL-1 receptor-related protein ST2 and induces T helper type 2-associated cytokines. Immunity 23:479-490.
Lefrançais E, Roga S, Gautier V, Gonzalez-de-Peredo A, Monsarrat B, Girard J P and Cayrol C. IL-33 is processed into mature bioactive forms by neutrophil elastase and cathepsin G. Proc. Natl. Acad. Sci. USA, 2012, 109:1673-1678 (* Co-senior authors)

1. An in vitro or ex vivo method for inducing proliferation of γδ T cells comprising treating said γδ T cells with a combination of a γδ Tcell activator and IL-33.

2. The in vitro or ex vivo method according to claim 1 wherein the γδ T cells are Vγ9Vδ2 T cells.

3. The in vitro or ex vivo method according to claim 1 comprising a preliminary step of isolating Peripheral Blood Mononuclear Cells from blood samples.

4. The in vitro or ex vivo method according to claim 1, wherein the γδ T cell activator is a phosphoantigen.

5. The in vitro or ex vivo method according to claim 4 wherein the phosphoantigen is BrHPP.

6-7. (canceled)

8. A culture medium comprising a γδ T cell activator and IL-33.

9. A culture medium according to claim 8 wherein the γδ Tcell activator is a phosphoantigen.

10. An in vitro method for inducing proliferation of γδ T cells wherein said method comprises the step of culturing γδ T cells with the culture medium as defined in claim 8.

11. The in vitro method according to claim 10 wherein the γδ T cells are Vγ9Vδ2 T cells.

12. A kit comprising: (i) at least one γδ Tcell activator and (ii) IL-33.

13. A pharmaceutical composition comprising a γδ T cell activator and IL-33 and a pharmaceutically acceptable carrier.

14. The pharmaceutical composition according to claim 13, wherein the γδ T cell activator is a phosphoantigen.

15. A method of treating infection, autoimmunity disease or a proliferative disease in a subject in need thereof, comprising administering to said subject a pharmaceutical composition comprising
a γδ T cell activator and IL-33; and
a pharmaceutically acceptable carrier.

16. The method of claim 15, wherein said proliferative disease is cancer.
